(19) United States
(12) Patent Application Publication
(10) Pub. No.: US 2018/0342317 A1
(43) Pub. Date: Nov. 29, 2018

(54) BARCODE DATABASE AND SOFTWARE UPDATE SYSTEM

(71) Applicant: BAYER HEALTHCARE LLC, Whippany, NJ (US)

(72) Inventors: Barry SKIRBLE, Allison Park, PA (US); Claus-Peter REISINGER, Berlin (DE)

(21) Appl. No.: 15/771,777

(22) PCT Filed: Sep. 27, 2016
(86) PCT No.: PCT/US2016/053904

§ 371 Date: Apr. 27, 2018

Related U.S. Application Data
(60) Provisional application No. 62/250,646, filed on Nov. 4, 2015.

(51) Int. Cl. G16H 40/20; G16H 70/40; G16H 20/17; G06K 19/06; G06F 17/30; G06K 19/02; G06K 7/10

(52) U.S. Cl. CPC G16H 40/20; G16H 70/40; G16H 20/17; G06K 7/10366; G06F 17/30289; G06K 19/025; G06K 19/06037

(57) ABSTRACT

Provided is a method of creating a record in a reference table contained in a database associated with a medical system. The method includes obtaining a new record barcode which includes pharmaceutical information embedded therein. The pharmaceutical information may include a pharmaceutical identifier and at least one of a pharmaceutical concentration, a pharmaceutical volume, a pharmaceutical expiration date, and a pharmaceutical lot and batch number. The method further includes scanning the new record barcode using a barcode reader associated with the medical system. Upon scanning the new record barcode, a processor associated with the database creates a reference table record in the reference table and populates the reference table record with the pharmaceutical information. Also provided is a system for implementing the above-described method.

### BACKGROUND OF THE INVENTION

### Field of the Invention

The present invention relates to systems and methods of updating a database, and more particularly to the use of barcode technology to update a database associated with a medical system with new medical information.

### Description of Related Art

In the medical field, there are competing interests between allowing information to flow freely and ensuring that such information remains secure and accurate. On the one hand, the free flow of information, such as through networked systems, allows for all of the nodes on the network to remain “up to date” by sending and receiving information in real-time. On the other, such network accessibility can create a portal by which data errors can quickly propagate throughout a system, jeopardizing the accuracy of the data across the entire network. Because of this, some institutions restrict the ways in which certain components receive information, even requiring that those components be updated “offline” by entering updated information locally.

One such component that may face such a restriction is a pharmaceutical database. A pharmaceutical database, as the name implies, is a database that contains information about a variety of pharmaceuticals that may be used by a point-of-care unit. By way of example, a radiology suite at a hospital may include a pharmaceutical database that contains information about the different contrast media that may be administered to a patient as part of an exam, such as the volume, concentration, manufacturer, expiration date, etc. In use, information contained in the database about a pharmaceutical is accessed to obtain a more complete understanding of the pharmaceutical being used as part of an exam. This information can then be included in an exam report, where it can be considered by a reviewing physician or quality control department. Traditionally, updating this database, and particularly adding new pharmaceuticals to the database, is accomplished in the same manner in which software is usually updated. This includes sending updates to the database over a network, having a technician manually key in information, or transferring information stored on a compact disc or USB drive. However, each of these techniques has inherent problems associated therewith. For one, as explained above, a networked database faces an increased risk of receiving corrupted or inaccurate information. Second, human error is a serious concern when manually entering data. Finally, the use of physical media can be expensive and difficult to quickly disseminate in a large hospital setting.

### SUMMARY

In one aspect, the invention is a method of creating a record in a reference table contained in a database associated with a medical system. The method includes obtaining a new record barcode comprising pharmaceutical information embedded therein, wherein the pharmaceutical information comprises a pharmaceutical identifier and at least one of a pharmaceutical concentration, a pharmaceutical volume, a pharmaceutical expiration date, and a pharmaceutical lot and batch number; and scanning the new record barcode using a barcode reader associated with the medical system, wherein scanning the new record barcode causes a processor associated with the database to create a reference table record in the reference table and populate the reference table record with the pharmaceutical information.

In some non-limiting embodiments, the pharmaceutical information includes at least the pharmaceutical identifier, the pharmaceutical concentration, the pharmaceutical volume, the pharmaceutical expiration date, and the pharmaceutical lot and batch number.

In certain non-limiting embodiments, the new record barcode is provided on a pharmaceutical container and the pharmaceutical information relates to a pharmaceutical contained in the pharmaceutical container. The pharmaceutical may be, for example, contrast media or a radiopharmaceutical.

In certain non-limiting embodiments, the new record barcode is provided on a sheet of printed paper. Obtaining the new record barcode may involve obtaining an electronic file from which the printed sheet of paper can be printed and printing the sheet of printed paper. The new record barcode may be a two dimensional barcode.

In certain non-limiting embodiments, the method can further include scanning a product barcode which is provided on a pharmaceutical container and includes the pharmaceutical identifier embedded therein. Scanning the product barcode then causes the processor to locate the reference table record using the pharmaceutical identifier, extract at least a portion of the pharmaceutical information from the reference table record, and populate a dataform with the at least a portion of the pharmaceutical information.

In some non-limiting embodiments, the medical system is a pharmaceutical injector system. Further, the database may not be accessible over a network.

In some non-limiting embodiments, scanning the new record barcode causes the processor to perform a validity verification function to authenticate the new record barcode.

In another aspect, the invention is a piece of printed media that includes a new record barcode, wherein the new record barcode comprises pharmaceutical information embedded therein, wherein the pharmaceutical information comprises a pharmaceutical identifier and at least one of a pharmaceutical concentration, a pharmaceutical volume, a pharmaceutical expiration date, and a pharmaceutical lot and batch number.

In certain non-limiting embodiments, the pharmaceutical information embedded in the new record barcode includes at least the pharmaceutical identifier, the pharmaceutical concentration, the pharmaceutical volume, the pharmaceutical expiration date, and the pharmaceutical lot and batch number.

In some non-limiting embodiments, the piece of printed media is provided on a pharmaceutical container and the pharmaceutical information relates to a pharmaceutical contained within the container.

In certain non-limiting embodiments, the new record barcode is a two-dimensional barcode.

In another aspect, the invention is a system that includes a barcode reader; a database containing a reference table comprising a plurality of records, wherein each record relates to a pharmaceutical product identified by a unique pharmaceutical identifier; a processor in communication with the barcode reader and the database; and a non-transitory, computer-readable storage medium in operable communication with the processor. The non-transitory, computer-readable storage medium contains one or more programming instructions that, when executed, cause the processor to receive pharmaceutical information obtained from a new record barcode using the barcode reader, wherein the pharmaceutical information comprises a pharmaceutical identifier and at least one of a pharmaceutical concentration, a pharmaceutical volume, a pharmaceutical expiration date, and a pharmaceutical lot and batch number; create a new reference table record in the database; and populate the new reference table record with the pharmaceutical information.

In certain non-limiting embodiments, the system further includes a piece of printed media comprising the new record barcode, wherein the new record barcode comprises the pharmaceutical information embedded therein.

In certain non-limiting embodiments of the system, the non-transitory, computer-readable storage medium further contains one or more programming instructions that, when executed, cause the processor to receive, from the barcode reader, an indication that a product barcode having embedded therein at least the pharmaceutical identifier has been read by the barcode reader; locate the reference table record using the pharmaceutical identifier; extract at least a portion of the pharmaceutical information from the reference table record; and populate a dataform with the at least a portion of the pharmaceutical information.

In some non-limiting embodiments, the system further comprises a pharmaceutical injector.

The foregoing and other objects, features, and advantages of the present invention become more apparent in light of the following detailed description of exemplary embodiments thereof, as illustrated in the accompanying drawings.

### BRIEF DESCRIPTION OF THE DRAWINGS

FIG. 1 is a flow chart of a process of creating a record in a reference table contained in a database and using the information in the record according to one non-limiting embodiment;

FIG. 2 is a schematic view of a system according to one non-limiting embodiment;

FIG. 3 is a representation of a set of database records according to one non-limiting embodiment;

FIG. 4 is a perspective view of a pharmaceutical container having a product barcode thereon according to one non-limiting embodiment; and

FIGS. 5A-5B illustrate a flowchart of a system in which a barcode tracking technology can be used according to one non-limiting embodiment.

### DESCRIPTION OF THE PREFERRED EMBODIMENTS

For purposes of the description hereinafter, spatial orientation terms, if used, shall relate to the referenced embodiment as it is oriented in the accompanying drawing figures or otherwise described in the following detailed description. However, it is to be understood that the embodiments described hereinafter may assume many alternative variations and configurations. It is also to be understood that the specific devices, features, and components illustrated in the accompanying drawing figures and described herein are simply exemplary and should not be considered as limiting. As used herein and in the appended claims, the singular forms “a,” “an,” and “the” include plural references unless the content clearly dictates otherwise.

As used herein, the terms “communication” and “communicate” refer to the receipt, transmission, or transfer of one or more signals, messages, commands, or other type of data. For one unit or device to be in communication with another unit or device means that the one unit or device is able to receive data from and/or transmit data to the other unit or device. A communication may use a direct or indirect connection, and may be wired and/or wireless in nature. Additionally, two units or devices may be in communication with each other even though the data transmitted may be modified, processed, routed, etc., between the first and second unit or device. For example, a first unit may be in communication with a second unit even though the first unit passively receives data, and does not actively transmit data to the second unit. As another example, a first unit may be in communication with a second unit if an intermediary unit processes data from one unit and transmits processed data to the second unit. For instance, data communicated from one component to another can pass through one or more nodes, which can serve as a local data collection and communication module performing functionality commonly associated with a networked system, such as “store and forward” and other low-level data collection, processing and communication functions. It will be appreciated that numerous other arrangements are possible. Throughout this description and in the figures, communication links from one component to another will be discussed and illustrated. For clarity, the arrows indicate the direction of the communication. The arrows may be understood to indicate separate, one-way communication links. Alternatively, they may indicate a single communication link that facilitates two-way communication. As would be appreciated by those skilled in the art, the communication link(s) may be a telephone line, a wireless communication link, or the Internet, among others.

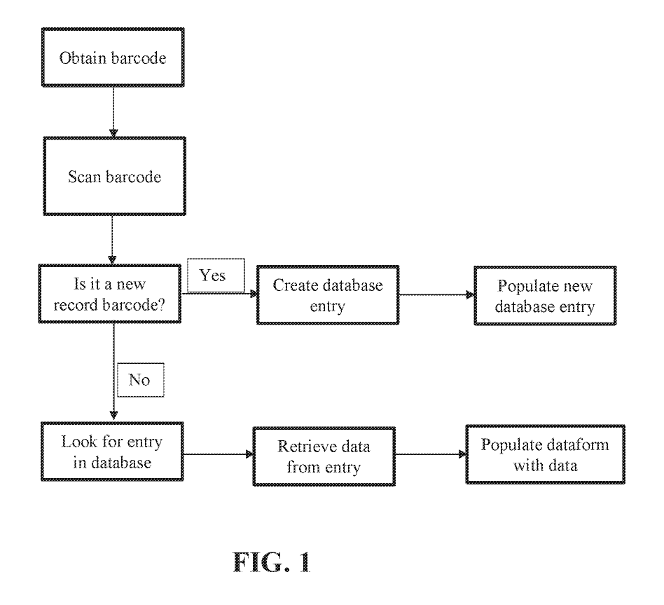

FIG. 1 is a flowchart illustrating various steps, stages, or phases of an exemplary process of creating a record in a reference table contained in a database associated with a medical system and using the information contained in the record to populate a dataform. The flowchart includes steps of obtaining a new record barcode which includes pharmaceutical information including at least a pharmaceutical identifier, scanning the new record barcode with a barcode reader associated with the medical system, and determining whether the barcode is a new record code (e.g., confirming that the new record barcode is indeed a new record barcode). If the system recognizes the barcode as a new record barcode, the process continues by initiating the creation of a new record in the reference table of the pharmaceutical database, and populating the new record with the pharmaceutical information encoded in the new record barcode. Alternatively, if the barcode that is scanned is not a new record barcode but is instead a product barcode which includes at least a pharmaceutical identifier encoded therein, the process includes determining whether there is an entry in the pharmaceutical database for the pharmaceutical identified by the encoded pharmaceutical identifier. If such a record exists, the process continues by recalling pharmaceutical information from the record and populating a dataform with such information. The flowchart is provided to facilitate understanding of the various embodiments of the invention that will now be disclosed. The various systems and methods described hereinafter are generally applicable to the generic process outlined in FIG. 1.

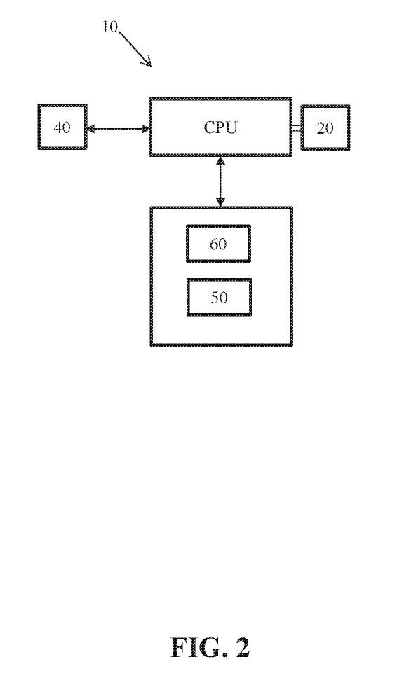

FIG. 2 illustrates a system 10 according to one embodiment. System 10 can include a barcode reader 20 for reading information from a variety of types of barcodes. System 10 can also include a pharmaceutical database 40 containing a reference table. System 10 may also include a processor 50 (or series of processors) in communication with both the barcode reader 20 and the database 40. System 10 may also include a non-transitory, computer readable storage medium 60 that is in operable communication with the processor 50 such that the processor 50 can execute instructions stored in the storage medium 60.

In some non-limiting embodiments, system 10 may include a single computer, a server computer, a combination of computers, or any other combination of hardware and/or software components. The individual units or components of system 10 may be localized or, in some embodiments, distributed among any number of hardware devices, local or remote, preferably in communication with one another. Further, each unit may itself be comprised of a distributed system, such as a series of servers and/or computers. In one non-limiting example, certain components of system 10, or even the entire system 10, may be incorporated into the software and hardware associated with medical imaging equipment (e.g., scanner and injector), such as the Certegra® Workstation product offered by Bayer HealthCare LLC. For example, the hardware and software associated with an injector device may also include barcode reader 20, pharmaceutical database 40, processor 50, and computer readable storage medium 60, or aspects thereof. In another example, certain components of system 10, or even the entire system 10, may be provided as part of an enterprise platform that performs other functions as well, such as the Radimetrics™ Enterprise Platform offered by Bayer HealthCare LLC. It will be appreciated that various other arrangements are possible.

The systems and methods described herein allow for offline updating of the pharmaceutical database 40. As will be apparent below, because the information that is used to create a new record in the pharmaceutical database 40 comes directly from the new record barcode, the pharmaceutical database need not be configured to receive such information over a network. Indeed, pharmaceutical database 40 need not even be “online” or otherwise accessible over a network since updates are input locally through the use of a new record barcode as described herein. This advantage is important for those organizations which, as a matter of policy, do not permit certain software and hardware components to remotely access updates across an intra- or inter-organization network, such as a local intranet or the World Wide Web.

With reference to FIG. 1, an embodiment of the process of using a barcode to create a database record will first be described. In a first step, an operator (such as a technician tasked with keeping the pharmaceutical database 40 up to date or with performing injections using pharmaceuticals) obtains a new record barcode. For purposes of the present disclosure, the term “barcode” is intended to encompass any optical machine-readable representation of data. The new record barcode can be in the form of a one dimensional linear barcode, such as the UPC/EAN code, that are widely used in supermarkets. Such one dimensional linear barcodes are characterized by data that is encoded along a single axis, in the widths of bars and spaces, so that such symbols can be read from a single scan along that axis, provided that the barcode is imaged with a sufficiently high resolution along that axis. The new record barcode may also be in the form of a number of one dimensional stacked barcodes, such as Code 49, as described in U.S. Pat. No. 4,794,239, which is incorporated by reference, and PDF417, as described in U.S. Pat. No. 5,340,786, which is incorporated by reference. Such stacked barcodes partition the encoded data into multiple rows, each including a respective one dimensional pattern, all or most all of which must be scanned and decoded, then linked together to form a complete message. Scanning still requires relatively high resolution in one dimension only, but multiple linear scans are needed to read the whole symbol. Preferably, however, new record barcode is in the form of a two-dimensional matrix barcode, which offers orientation-free scanning and greater data densities and capacities than one-dimensional barcodes. Two-dimensional matrix codes encode data as dark or light data elements within a regular polygonal matrix, accompanied by graphical finder, orientation and reference structures. When scanning two-dimensional matrix codes, the horizontal and vertical relationships of the data elements are recorded with about equal resolution. Other forms of barcodes, both currently existing and developed in the future, including three-dimensional barcodes, may also be used with the systems and methods disclosed herein. Barcodes are also described in, for example, U.S. Pat. No. 7,546,954, which is incorporated herein by reference.

While it is envisioned that the system and methods described herein could be implemented using an RFID tag or other data storage means in place of a barcode, barcodes provide several advantages over RFID tags in this environment. For one, barcodes are much more cost effective than RFID tags. Barcodes need only to be visually displayed in some manner, such as through printing on a sheet of paper or displayed on a computer screen. RFID tags require programming of the tag which itself includes an integrated circuit for storing information, modulating RF signals, and collecting power as well as an antenna for communicating with an RFID reader. In addition, barcodes generally occupy less real estate which allows them to be more easily placed on a small pharmaceutical container surface, such as a vial or syringe. Still further, barcodes do not utilize RF communication. In a medical setting, there are often many devices communicating in the RF range, and thus a greater chance that interference occurs with communications operating in this range. Additionally, barcodes cannot be as easily reconfigured or reprogrammed as an RFID tag. Therefore, the risk of tampering with or modifying a barcode so that the barcode delivers incorrect information is less than with an RFID tag.

Encoded within the new record barcode is a variety of information pertinent to the new record to be created in the pharmaceutical database 40. Non-limiting examples of the information encoded within the new record barcode include a pharmaceutical identifier, a pharmaceutical concentration, a pharmaceutical volume, a pharmaceutical expiration date, and a pharmaceutical lot and batch number. Some or all of this information can be encoded in the new record barcode. Additional information can be encoded in the new record barcode as well. It is also possible that some of this information is encoded in the new record barcode while other is entered manually. Preferably, at least the pharmaceutical identifier and the pharmaceutical lot and batch number are encoded in the new record barcode.

The pharmaceutical identifier serves to identify the pharmaceutical to which the record to be created will pertain. Pharmaceutical identifier can be, for example, a product number, such as a universal product code (“UPC”) or a global trade item number (“GTIN”). Commonly, in the pharmaceutical industry, each pharmaceutical that is commercially available is assigned a UPC or GTIN which is universally used when referencing that particular pharmaceutical during the manufacture, purchase, shipping, and/or administration thereof. The pharmaceutical concentration and pharmaceutical volume, as the name implies, constitute the concentration and volume associated with the particular pharmaceutical, respectively. Again, it is typically the case that a pharmaceutical with a particular GTIN will have a set concentration and volume. Changes to either the concentration or volume will require the assignment of a new GTIN, even if the chemical makeup of the pharmaceutical remains unchanged. The pharmaceutical expiration date and lot and batch number are, again, specific to the pharmaceutical and are, again, generally specific to a particular GTIN.

The new record barcode may also contain encoded information that identifies the barcode as a new record barcode. As explained herein, a new record barcode is one that will be understood by the system to initiate the creation of a new record in the pharmaceutical database 40. Thus, inclusion in the barcode itself of some encoded information that identifies the barcode as a new record barcode will allow the system to recognize the barcode as such and initiate the process of creating a new database record. Alternatively, the user may recognize the barcode as a new record barcode due to some other indicia on the media on which the barcode appears and may signify to the system that the barcode is a new record barcode. For example, the user may enter a command signifying that the next barcode to be read is a new record barcode and, thus, the system should use the information received from barcode reader 20 to create a new record. As another alternative, the system may treat the barcode as a new record barcode if the barcode does not identify a pharmaceutical that is recognized by the system, such as a pharmaceutical that is not already in the pharmaceutical database. For example, if a pharmaceutical identifier encoded within a barcode does not match with any pharmaceutical identifier already in the pharmaceutical database 40, the system can treat the barcode as a new record barcode and initiate the process of creating a new database record based on information encoded in the barcode.

The new record barcode can be obtained in a variety of different ways. In one example, the new record barcode can be provided to the operator as a hard copy print out, such as on a sheet of paper. In another non-limiting embodiment, the new record barcode can be provided in an electronic format, such as on the display screen of a PDA or similar type of device capable of displaying a barcode. In yet another non-limiting embodiment, the new record barcode can be provided on a pharmaceutical container, such as on the label thereof. Regardless of what form the new record barcode is ultimately provided, the new record barcode can be disseminated through the Internet or other electronic network, including by email, as an electronic file. The file, once downloaded onto a computer, can be opened to enable the new record barcode to be visually displayed on a screen, such as a PDA screen, or printed, such as by a printer that is in communication with a computer that has received the electronic file. The new record barcode can also be printed and sent through the physical mail and/or intra-office mail or included in the product packaging.

Returning to FIG. 1, once the new record barcode is obtained, it is scanned by the barcode reader 20 associated with system 10. Barcode reader 20 can be any barcode reader 20 known in the art that is capable of reading optical barcodes. Such barcode readers are generally known and are discussed in, for example, U.S. Pat. Nos. 6,223,988, 7,137,555, 7,320,431, and 8,602,309, the entire contents of each of which are incorporated by reference. Preferably, barcode reader 20 can read both one dimensional and two dimensional barcodes. However, it is also possible to use multiple barcode readers 20 each with a different capability. After scanning the barcode, barcode reader 20 communicates encoded information within barcode to the processor 50. Communication between the barcode reader 20 and processor 50 may be through any available communication medium, including both wired and wireless means, and may use any available communication protocol, such as Bluetooth or Wi-Fi.

Processor 50, upon receiving data from barcode reader 20, may first determine whether the barcode is a new record barcode. This can be done by determining the presence (or absence) of a new record code encoded within the barcode. Alternatively, as described above, processor 50 may already be aware that the barcode is a new record barcode by way of an input provided by the operator. If it is determined that the barcode is a new record barcode, processor 50 initiates the process of creating a new record in the pharmaceutical database 40.

Processor 50 may also perform one or more validity verification functions to authenticate the new record barcode. This could include comparing certain data encoded within the new record barcode with preprogrammed data that already resides on system 10 in order to determine if the new record barcode is authentic. This process could involve decrypting an encrypted number encoded in the barcode with a key resident on the system 10 and comparing the decrypted number against an anticipated value where the anticipated value may also be encoded in the new record barcode. Performing a validity verification function can help ensure that the new record barcode originates from a trusted source and also contains accurate information so that only correct information is loaded in pharmaceutical database 40. The validity verification function can also ensure that the new record barcode has not been tampered with or otherwise reconfigured (including through data corruption) since it was generated.

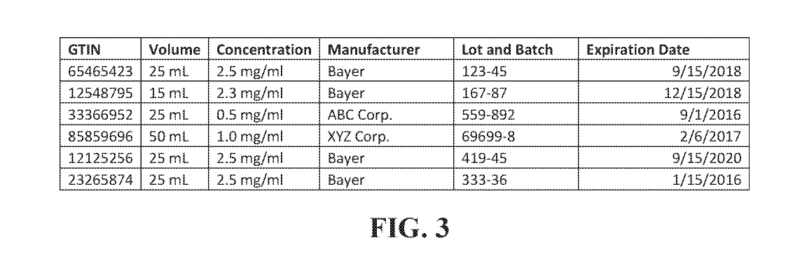

The pharmaceutical database 40 constitutes one or more data structures configured to store pharmaceutical data. In some non-limiting embodiments, the pharmaceutical database 40 may include data structured as one or more tables, trees, arrays, objects, and/or other like data structures. The data may be arranged by pharmaceutical, cell type, pharmaceutical identifier, therapeutic use, protocol name, and/or the like. It will be appreciated that, with some forms of structured data, the data may be arranged in any number of ways depending on how it is queried. In non-limiting embodiments utilizing an object-oriented database, the pharmaceuticals themselves may be individual objects with various attributes and parameters. It will be appreciated that any other data storage methods and/or data structures may be used. FIG. 3 illustrates a representative set of records that can be stored in pharmaceutical database 40. Creation of the new record entry proceeds according to normal database record creation procedures available in commercially available software. The header of the record can based on the pharmaceutical identifier, and may be the pharmaceutical identifier itself, such as the GTIN or UPC.

Once the record is created, the record is also populated with information that is encoded in the new record barcode about the particular pharmaceutical that is the subject of the record. This can include such information as the concentration and/or volume of the pharmaceutical, the expiration date, the manufacture date, the batch and lot number, the manufacturer's name, etc. As a result, following the creation and population of the new record, pharmaceutical database 40 now contains a new record for a new pharmaceutical, where this new record includes information about the pharmaceutical such as its GTIN, volume, concentration, batch and lot number, manufacturer, expiration date, and manufacturer's name. This information can be maintained in the pharmaceutical database 40 and some or all of it can be recalled upon request.

Returning again to FIG. 1, the process of utilizing a new record created in the pharmaceutical database will now be described. In this process, a product barcode is received by the operator. The product barcode, like the new record barcode, can be a one-dimensional linear barcode, a stacked one-dimensional barcode, or a two-dimensional matrix style barcode. Like the new record barcode, the product barcode contains at least a pharmaceutical identifier. However, unlike the new record barcode, the product barcode need not contain a new record code or the same amount of pharmaceutical information as new record barcode for reasons that will be apparent below. Thus, since the product barcode need not contain as much information as new record barcode, a one-dimensional barcode may be sufficient. The product barcode is used in much the same way as a UPC at a grocery store. In particular, the product barcode is used to allow the system to recognize the pharmaceutical contained within the pharmaceutical container, based on a pharmaceutical identifier encoded in the product barcode, and to recall information about that pharmaceutical from the pharmaceutical database 40, using the pharmaceutical identifier to locate the record within the database.

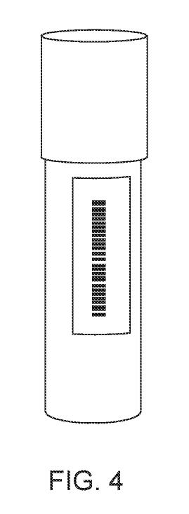

A product barcode is generally provided on or in association with a pharmaceutical container containing a pharmaceutical that is to be delivered to a patient as part of a medical procedure. Non-limiting examples of such pharmaceutical containers include vials and syringes. Non-limiting examples of pharmaceuticals include contrast media and radiopharmaceuticals. In one non-limiting embodiment, the product barcode is printed on a label that is affixed to the outside of a pharmaceutical container, an example of which is illustrated in FIG. 4. In another non-limiting embodiment, the product barcode is printed on packaging in which a pharmaceutical container is placed. In yet another non-limiting embodiment, the product barcode is printed on literature that is provided in conjunction with the pharmaceutical container.

Referring again to FIG. 1, the product barcode, once obtained, is scanned by the barcode reader 20. After scanning the product barcode, barcode reader 20 communicates information read from the product barcode to the processor 50. Communication between the barcode reader 20 and processor 50 may be through any available communication medium, including both wired and wireless means, and may use any available communication protocol, such as Bluetooth or Wi-Fi. This information should include at least a pharmaceutical identifier.

Processor 50, upon receiving data from barcode reader 20, may first determine whether the barcode is a product barcode or a new record barcode. This can be done by determining the presence (or absence) of a new record code encoded within the barcode. Alternatively, processor 50 may already be aware that the barcode is a product barcode by way of an input provided by the operator. In still yet another alternative, processor 50 uses the data received from barcode, including the pharmaceutical identifier, and determines whether the pharmaceutical database includes a record corresponding to the pharmaceutical identifier received. If there is a match, then the processor 50 recognizes that the barcode is a product barcode and is not, for example, a new record barcode.

If it is determined that the barcode is a product barcode, processor 50 initiates the process of locating the record in the pharmaceutical database 40 which corresponds to the pharmaceutical identifier encoded in the product barcode and extracting pharmaceutical information from the record in the pharmaceutical database 40 about the pharmaceutical. Traditional database search and recall techniques can be used to accomplish these stages of the process.

The information, once extracted, can be used to populate the entries in a dataform. In one non-limiting embodiment, the information from the record can be used to create a detailed report about the pharmaceutical being used as part of a medical procedure. By way of example, a technician performing a contrast media injection procedure on a patient may receive a vial that contains contrast media. On the container is a printed label that includes a product barcode. Encoded within the product barcode is a pharmaceutical identifier. The pharmaceutical database 40 already includes a record corresponding to the particular pharmaceutical identifier encoded in the product barcode. The pharmaceutical database 40 may include this record because a new record was previously created for this pharmaceutical identifier according to the process described above. Either in advance of the injection procedure or subsequent thereto, the technician scans the product barcode. Upon scanning the product barcode, the system identifies the pharmaceutical identifier encoded within the product barcode and extracts from the pharmaceutical database 40 information about the pharmaceutical, in this case contrast media, which corresponds to the pharmaceutical identifier. Once extracted, the information is then included in a report about the injection procedure. Thus, the report can include information about the contrast volume, concentration, expiration date, manufacturer, and lot and batch number of the pharmaceutical, all of which has been extracted from the pharmaceutical database. This information can be automatically populated in the report, alleviating any need for the technician to manually enter the information into the report, thereby greatly simplifying the task of creating the report and limiting the risk of data entry errors or omissions.

An example of utilizing the invention is now briefly described. First, a customer obtains a pharmaceutical product information sheet of two new products either in the product box, by email, or over the Internet. The new products are Gadovist 1.0 MMO/ML Injectable and Gadovist 2.0 MMO/ML Injectable. The sheet has at least one barcode associated with each new product displayed thereon. The customer scans the barcodes with the provided barcode reader described above. The system, upon accepting the barcodes, will either add a new record into the pharmaceutical reference table in the database or will update an existing record in the database with information about these products. The customer will now be able to select and utilize this data without having to first hand-enter the data. This will save the customer time as well as guaranteeing the correctness of the data being added to the system. In another example, this method could be used for updating catheter gauges. Instead of a customer hand-entering data into the system, the customer could easily scan a barcode present on a data sheet that is sent to the customer with information on all of the catheter gauges that are supported by a particular injection system.

In certain non-limiting embodiments, system 10 can be paired with or part of an injection system or fluid delivery system. The term “injection system,” as used herein, refers to one or more hardware and/or software components, systems, modules, and/or the like used to deliver fluid to a patient, an animal, or a test receptacle. It will be appreciated that the various components and/or modules of the injection system may be included in a single housing, may be separated, and/or may be arranged in any other operable way. The term “fluid delivery system,” as used herein, refers to a portion of an injection system, or separate components or devices in communication with an injection system, which deliver fluid to patients, animals, or test receptacles, and/or control fluid delivery. In some non-limiting embodiments, the fluid delivery system may include a syringe motor, actuator, interface, one or more pumps, one or more pump cartridges, one or more ports for receiving pump cartridges, tubing or other fluid paths, and/or the like. Examples of injection and fluid delivery systems that can be used with system 10 include, but are not limited to, those described in U.S. Pat. App. Pub. No. 2012/0123257, U.S. Pat. App. Pub. No. 2008/0086087, and WIPO Pub. No. WO/2012/155035, all of which are hereby incorporated by reference in their entirety. The fluid delivery systems may include various components such as, but not limited to, those discussed in U.S. Pat. Nos. 8,337,456 and 8,147,464, which are also hereby incorporated by reference in their entirety.

While system 10 has been described herein in connection with updating a pharmaceutical database 40 with information about pharmaceuticals, the general principles and techniques can be applied in other settings as well. In one non-limiting example, the system and process described herein can be used to update a database of injection protocols. In this example, an operator obtains a protocol barcode in one of the manners described above with respect to the new record barcode. For example, an electronic file can be downloaded and opened to display a protocol barcode which can then be printed into hard copy form or displayed on a PDA or similar device. The protocol barcode can contain details about an injection protocol, including flow rates, injection times, concentrations, pressures, and other parameters. The protocol barcode can also contain a protocol identifier. Once obtained, the protocol barcode can be scanned, and a new record can be created in a protocol database according to a process as described above for the creation of a new record in a pharmaceutical database. A technician can then recall the details of the protocol by searching for the protocol identifier in the protocol database. Once located, the parameters associated with the protocol can be automatically populated into an injection controller so that an injection procedure can proceed according to the protocol. The process of locating a particular protocol can also occur automatically by scanning another barcode that has encoded therein the protocol identifier, which triggers the system to locate the protocol record associated with that protocol identifier and populate the injector controller with the protocol details. Such a barcode could appear, for example, on a prescription for an injection procedure, on a patient's chart, or on a container holding the pharmaceutical to be used for the injection. Thus, in one non-limiting embodiment, a technician may review a patient's chart in advance of the injection procedure and use the barcode scanner 20 to scan a barcode printed on the chart, whereby the system locates in the protocol database the protocol identified from the scan of the barcode and communicates the parameters for that protocol to the injection controller so the protocol can be used to perform the injection procedure.

The systems and methods described herein could also be used to update an existing record in the pharmaceutical database 40 rather than to create a new record. In such an embodiment, the new record barcode could instead be an update record barcode. The system 10 could recognize this barcode as an update record barcode, locate the record to be updated, and make the necessary changes to the record based on information and instruction provided in the update record barcode. The update record barcode could also include a code that would indicate the barcode is intended to be used to update an existing record in the pharmaceutical database 40. For example, the update record barcode could include a pharmaceutical identifier as well as a replacement value for the “pharmaceutical volume” entry. Upon scanning the update record barcode, the system would recognize the barcode as an update record barcode, would locate the record to be updated, and would replace the “pharmaceutical volume” entry for that record with the new value that is encoded in the update record barcode.

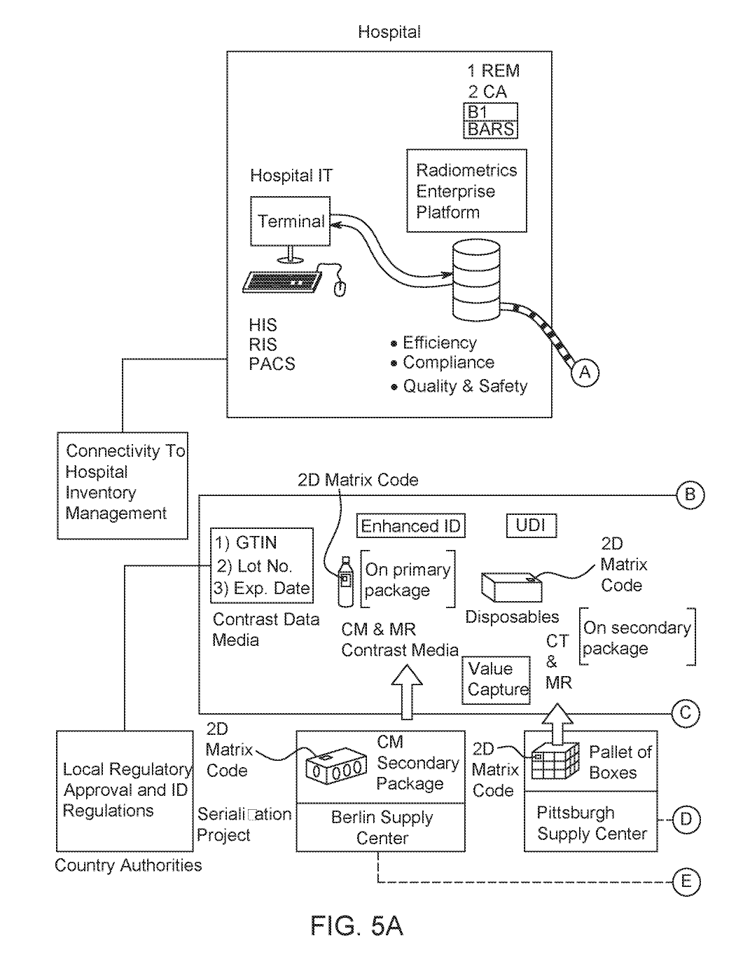

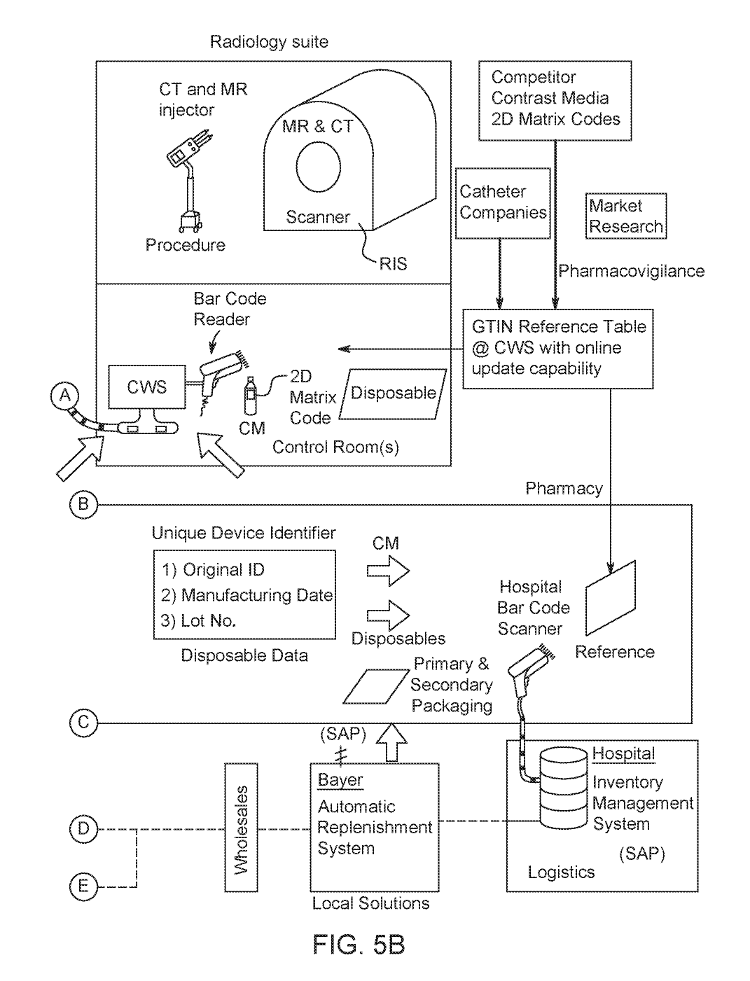

Turning to FIGS. 5A-5B, what is shown is a flowchart of a system in which barcode tracking technology can be used according to one non-limiting embodiment. The system disclosed in FIGS. 5A-5B includes various components, locations, and business units that may benefit from the barcode technology discussed herein. For example, the flowchart identifies different supply centers (“Berlin Supply Center” and “Pittsburgh Supply Center”) where boxes of pharmaceuticals are manufactured, packaged, and/or supplied. These boxes, as well as the individual units therein, could have displayed on the outside thereof a product barcode like is described above to identify the pharmaceuticals contained therein. These boxes, as well as the individual units therein, could also have displayed a new record barcode if the pharmaceutical contained in the boxes are ones that are not yet in a pharmaceutical database 40. The flowchart also identifies a “Pharmacy” which could receive the pharmaceutical supply from the supply centers. Various functions related to the barcode technology described herein could be implemented at the Pharmacy. For one, the Pharmacy could be the location where the new record barcodes are first scanned so as to create a new record at the Pharmacy level for the new pharmaceutical. The Pharmacy can also represent the location where a new record barcode is created, such as by physically printing and attaching a barcode to the boxes supplied from the Supply Centers or the units thereof. To this end, the Pharmacy can be in communication with a database storing pharmaceutical information that can be encoded into a new record barcode, such as the GTIN. Assignment of a GTIN may be regulated by a local regulatory agency that sets the GTIN for a particular pharmaceutical. As depicted in FIGS. 5A-5B, the Pharmacy may communicate with this agency. The Pharmacy can also be in communication with one or more inventory management systems that can track the pharmaceutical usage based on, for example, information drawn from a barcode scan performed at the point of care. The inventory management system can be used to determine, based on available quantity, existing orders, and/or historical usage rates, when the supply should be replenished. As depicted in FIGS. 5A-5B, inventory management system may be in communication with an automatic replenishment system which may be in communication with wholesalers and/or the supply centers mentioned previously.

FIGS. 5A-5B also depicts the flow of information between the point of care unit, in this case a radiology suite, the Pharmacy, and the larger hospital network. In FIGS. 5A-5B, the Pharmacy can be in communication with a GTIN reference table, which is one embodiment of the pharmaceutical database 40 discussed above. Records in the GTIN reference table can be based on information received or entered at the Pharmacy. The GTIN reference table can be in communication with the control room of the radiology suite, where a computer equipped with a barcode reader is present. The computer can be a Certegra® Workstation product offered by Bayer HealthCare LLC. The Certegra® Workstation product can be in communication with various informational systems in the hospital, including the Radimetrics™ Enterprise Platform offered by Bayer HealthCare LLC, along with the radiology information system (RIS), hospital information system (HIS), and picture archiving and information system (PACS). When performing a scan in the radiology suite, a scan of a product barcode contained on the contrast media container can initiate the process described above of recalling information from the GTIN database which can then be included with a report that is generated from the scan. The report can then be communicated to the other systems within the hospital for storage, retrieval, and further processing.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A method of creating a record in a reference table contained in a database associated with a medical system, comprising:
obtaining a new record barcode comprising pharmaceutical information embedded therein, wherein the pharmaceutical information comprises a pharmaceutical identifier and at least one of a pharmaceutical concentration, a pharmaceutical volume, a pharmaceutical expiration date, and a pharmaceutical lot and batch number; and
scanning the new record barcode using a barcode reader associated with the medical system,
wherein scanning the new record barcode causes a processor associated with the database to create a reference table record in the reference table and populate the reference table record with the pharmaceutical information.

2. The method of claim 1, wherein the pharmaceutical information comprises at least the pharmaceutical identifier, the pharmaceutical concentration, the pharmaceutical volume, the pharmaceutical expiration date, and the pharmaceutical lot and batch number.

3. The method of claim 1, wherein the new record barcode is provided on a pharmaceutical container and the pharmaceutical information relates to a pharmaceutical contained in the pharmaceutical container.

4. The method of claim 3, wherein the pharmaceutical is one of contrast media and a radiopharmaceutical.

5. The method of claim 1, wherein the new record barcode is provided on a sheet of printed paper.

6. The method of claim 5, wherein obtaining the new record barcode comprises printing the sheet of printed paper.

7. The method of claim 6, wherein obtaining the new record barcode further comprises obtaining an electronic file from which the printed sheet of paper can be printed.

8. The method of claim 1, further comprising scanning a product barcode,
wherein the product barcode is provided on a pharmaceutical container,
wherein the product barcode comprises the pharmaceutical identifier embedded therein, and
wherein scanning the product barcode causes the processor to locate the reference table record using the pharmaceutical identifier, extract at least a portion of the pharmaceutical information from the reference table record, and populate a dataform with the at least a portion of the pharmaceutical information.

9. The method of claim 1, wherein the medical system is a pharmaceutical injector system.

10. The method of claim 1, wherein the database is not accessible over a network.

11. The method of claim 1, wherein the new record barcode is a two-dimensional barcode.

12. The method of claim 1, wherein scanning the new record barcode further causes the processor to perform a validity verification function to authenticate the barcode.

13. A piece of printed media, comprising a new record barcode, wherein the new record barcode comprises pharmaceutical information embedded therein, wherein the pharmaceutical information comprises a pharmaceutical identifier and at least one of a pharmaceutical concentration, a pharmaceutical volume, a pharmaceutical expiration date, and a pharmaceutical lot and batch number.

14. The piece of printed media of claim 13, wherein the pharmaceutical information comprises at least the pharmaceutical identifier, the pharmaceutical concentration, the pharmaceutical volume, the pharmaceutical expiration date, and the pharmaceutical lot and batch number.

15. The piece of printed media of claim 13, wherein the piece of printed media is provided on a pharmaceutical container and the pharmaceutical information relates to a pharmaceutical contained within the container.

16. The piece of printed media of claim 13, wherein the new record barcode is a two-dimensional barcode.

17. A system, comprising:
a barcode reader;
a database containing a reference table comprising a plurality of records, wherein each record relates to a pharmaceutical product identified by a unique pharmaceutical identifier;
a processor in communication with the barcode reader and the database;
a non-transitory, computer-readable storage medium in operable communication with the processor, wherein the non-transitory, computer-readable storage medium contains one or more programming instructions that, when executed, cause the processor to:
receive pharmaceutical information obtained from a new record barcode using the barcode reader, wherein the pharmaceutical information comprises a pharmaceutical identifier and at least one of a pharmaceutical concentration, a pharmaceutical volume, a pharmaceutical expiration date, and a pharmaceutical lot and batch number;
create a new reference table record in the database; and
populate the new reference table record with the pharmaceutical information.

18. The system of claim 17, further comprising a piece of printed media comprising the new record barcode, wherein the new record barcode comprises the pharmaceutical information embedded therein.

19. The system of claim 17, wherein the non-transitory, computer-readable storage medium further contains one or more programming instructions that, when executed, cause the processor to:
receive, from the barcode reader, an indication that a product barcode having embedded therein at least the pharmaceutical identifier has been read by the barcode reader;
locate the reference table record using the pharmaceutical identifier;
extract at least a portion of the pharmaceutical information from the reference table record; and
populate a dataform with the at least a portion of the pharmaceutical information.

20. The system of claim 17, further comprising a pharmaceutical injector.
